(19) United States
(12) Patent Application Publication
(10) Pub. No.: US 2016/0106381 A1
(43) Pub. Date: Apr. 21, 2016

(54) ULTRASOUND PROBE WITH TACTILE INDICATOR

(71) Applicant: General Electric Company, Schenectady, NY (US)

(72) Inventors: Svein Arne Aase, Trondheim (NO); Leif Peder Schmedling, Toensberg (NO)

(21) Appl. No.: 14/519,106

(22) Filed: Oct. 20, 2014

(51) Int. Cl. A61B 5/00; A61B 17/34; A61B 8/00

(52) U.S. Cl. CPC A61B 5/7455; A61B 8/4455; A61B 8/429; A61B 17/3403; A61B 2017/3413

(57) ABSTRACT

A method and apparatus actuate a tactile indicator on a hand-contacted surface of the handheld ultrasound probe based upon a parameter of the handheld ultrasound probe.

### BACKGROUND

Ultrasound systems comprise ultrasound scanning devices, such as ultrasound probes. The ultrasound probes are connected to an ultrasound system for controlling the operation of the probes. Such ultrasound probes comprise a scan head having a plurality of transducer elements (e.g., piezoelectric crystals), which may be arranged in an array. The transducers are used to perform various different ultrasound scans such as different imaging of a volume or body. During a scan of a volume or body, the ultrasound system drives the transducer elements within the array based upon the type of scan to be performed.

During ultrasound scanning, the caretaker must often concurrently visually monitor and evaluate a wide variety of different parameters. For example, the caretaker must often ensure that there is acceptable acoustic contact between the ultrasound probe in the anatomy or object being scanned. In many cases, the caretaker must also locate or orient the probe with respect to an intended target such as a desired imaging plane location, a needle or the like. Visually monitoring and evaluating such a wide variety of different parameters at the same time can be challenging, may result in poor image quality (such as resolution and/or signal to noise ratio) and may prolong the time consumed by the scan.

### BRIEF DESCRIPTION OF THE DRAWINGS

FIG. 1 is a schematic diagram of an example ultrasound system that provides tactile feedback.

FIG. 2 is a flow diagram of an example method for providing tactile feedback regarding acoustic contact of an ultrasound probe.

FIG. 3 is a diagram illustrating an example spatial frequency response of an imaging system.

FIG. 4 is a diagram illustrating an example lateral frequency response for a two-way aperture function.

FIG. 5 is a diagram illustrating various example a picture contacts and corresponding lateral spectrums for a linear phase array probe.

FIG. 6 is a diagram illustrating various example aperture contacts and corresponding lateral spectrums for a two-dimensional matrix probe.

FIG. 7 is a schematic diagram of another example ultrasound system that provides tactile feedback regarding acoustic contact of an ultrasound probe with an object or anatomy being examined.

FIG. 8 is a schematic diagram of yet another example ultrasound system that provides tactile feedback regarding acoustic contact of an ultrasound probe with an object or anatomy being examined.

FIG. 9 is a schematic diagram of an example tactile indicator for the ultrasound system of FIG. 8.

FIG. 10 is a schematic diagram of another example tactile indicator for the ultrasound system of FIG. 8.

FIG. 11 is a schematic diagram of another example tactile indicator for the ultrasound system of FIG. 8.

FIG. 12 is a fragmentary perspective view of another example tactile indicator for the ultrasound system of FIG. 8 being manually contacted.

FIG. 13 is a fragmentary sectional view of a portion of the tactile indicator of FIG. 12.

FIG. 14 is a schematic diagram of another example ultrasound system that provides tactile feedback regarding acoustic contact of an ultrasound probe with an object or anatomy being examined.

FIG. 15 is a perspective view of an example probe of the ultrasound system of FIG. 14.

FIG. 16 is a fragmentary perspective view of the probe of FIG. 15.

FIG. 17 is a schematic diagram of another example ultrasound system providing tactile feedback.

FIG. 18 is a flow diagram of an example method for providing tactile feedback regarding relative positioning of a target.

### DETAILED DESCRIPTION OF EXAMPLES

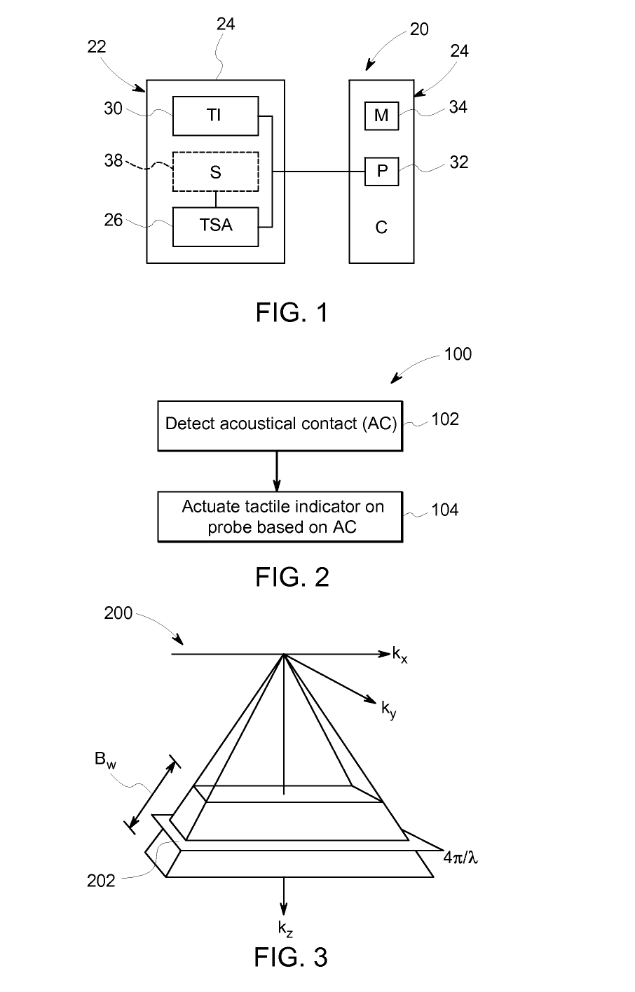

FIG. 1 schematically illustrates an example ultrasound system 20. As will be described hereafter, system 20 provides tactile feedback to a caretaker, reducing the number of parameters which must be visually monitored and evaluated by the caretaker. In the example illustrated, system 20 provides tactile feedback regarding acoustic contact between a handheld ultrasound probe and the volume or body being scanned.

Proper acoustical or acoustic contact between the probe and the volume or body being scanned facilitates the generation of images having acceptable resolution. During ultrasound image formation in phased array probes, a large part of the aperture of the probe is used for steering and focusing along each beam direction. As a result, a reduction of acoustical contact for portions of the probe surface reduces the effective aperture and results in poor image resolution, sometimes observed as a smearing in the lateral direction of the image. Reduced signal to noise ratios is caused by reduced power transmitted into the body by the reduced effective aperture. As a result, fewer second harmonic signals are created.

Reduced or poor acoustical contact may arise from multiple factors. For example, poor acoustical skin contact may result from an insufficient amount of contact gel being used, especially when the probe surface is not parallel to the skin surface. In cardiac imaging, achieving proper probe contact with the patient skin may be difficult due to the narrow acoustic window between the patient's ribs. To ensure good probe placement and acoustic contact for a given imaging application, a caretaker must often view a display screen, move the probe and adjust the probe settings.

During scanning, the experienced caretaker may recognize the occurrence of poor lateral image resolution and may adjust the probe position to improve image occurrence of poor lateral image resolution (e.g., adjust the probe position to improve image quality). However, such adjustment is a time consuming and challenging process. For less experienced caretakers, identifying acoustical contact issues and performing operations to correct for the issues is even more challenging, resulting in less than acceptable images (e.g., inability to perform proper diagnosis based on the image).

By providing the caretaker with tactile feedback regarding acoustical contact between the probe and the scanned body, system 20 facilitates better acoustical contact to facilitate generation of ultrasound images having improved quality or resolution. System 20 comprises probe 22 and controller 24. Probe 22 comprises a manually held or handheld device or instrument having a surface 24 to be manually contacted by a caretaker's hand while the hand is manipulating probe 22. As further schematically show FIG. 1, probe 22 additionally comprises transducer sensing area 26 and tactile indicator 30.

Transducer sensing area (TSA) 26 comprises that portion of probe 22 to be positioned against or in proximity to the body being scanned, providing acoustical contact with the body being scanned. In one implementation such “acoustical contact” is facilitated by contact gel between the skin of a patient and the transducer sensing area 26 of probe 22. In one implementation, transducer sensing area 26 comprises quartz crystals, piezoelectric crystals, that change shape in response to the application electrical current so as to produce vibrations or sound waves. Likewise, the impact of sound or pressure waves upon such crystals produce electrical currents. As a result, such crystals are used to send and receive sound waves. In one implementation, transducer sensing area 26 comprises a plurality sensing portions, such as a plurality of transducer sub apertures or contact apertures. In some implementations, transducer sensing area 26 may additionally include a sound absorbing substance to eliminate back reflections from the probe itself and an acoustic lens to focus emitted sound waves.

Tactile indicator 30 comprises one or more devices that provide tactile feedback to the person gripping or holding probe 22. Such tactile feedback indicates acoustical contact between transducer sensing area 26 and the body or anatomy being examined. In one implementation, such tactile feedback indicates a general quality of acoustical contact between transducer sensing area 26 and the body or anatomy being examined for the overall area of trenches a sensing area 26. In another implementation, such tactile feedback indicates quality of acoustical contact for different specific regions or portions of tactile sensing area 26. For example, in one implementation, such tactile feedback indicates which regions or portions of transducer sensing area 26 have acoustical contact satisfying a predefined threshold and which regions or portions of transducer sensing area 26 have acoustical contact that does not satisfy the predetermined threshold. In yet another implementation, such tactile feedback indicates a different quality or degree of acoustical contact for each of the portions of transducer sensing area 26. For example, such tactile feedback may indicate that a first portion has a first degree of acoustical contact, a second portion has a second degree of acoustical contact better than the first degree of acoustical contact and a third portion has a third degree of acoustical contact better than the second degree of acoustical contact.

In one implementation, tactile indicator 30 comprises one or more haptic devices which provide feedback in the form of touch by applying forces, vibrations or motions to the caretaker's hand that is gripping or holding probe 22. For example, in one implementation, tactile indicator 30 comprises a two-dimensional array, a row or a matrix of projections, pins, rods or bumps that are selectively raised and lowered to different heights above the underlying substrate or surface of probe 24 based upon determined acoustical contact of transducer sensing area 26. In another implementation, tactile indicator 30 comprises one or more individual vibration motors which produce a vibration sensation at different locations along surface 24. In still other implementations, tactile indicator 30 provides tactile feedback in other manners such as through temperature variations, wherein proportions of surface 24 are heated (or cooled) to different temperatures based upon acoustical contact of transducer sensing area 26 with the body or anatomy being examined.

Controller 24 comprises one or more processing units 32 and associated memory 34 that control provision of acoustic contact feedback to the caretaker through tactile indicator 30. For purposes of this application, the term “processing unit” shall mean a presently developed or future developed processing unit that executes sequences of instructions contained in a memory, such as memory 34. Execution of the sequences of instructions causes the processing unit to perform steps such as generating control signals.

Memory 34 comprises a non-transitory computer-readable medium upon which are stored code, software or other programmed logic defining the sequences of instructions for controlling operation of tactile indicator 30. Memory 34 may be in the form of a random access memory (RAM) for execution by the processing unit from a read only memory (ROM), a mass storage device, or some other persistent storage. In other embodiments, hard wired circuitry may be used in place of or in combination with software instructions to implement the functions described. For example, controller 24 may be embodied as part of one or more application-specific integrated circuits (ASICs). Unless otherwise specifically noted, the controller is not limited to any specific combination of hardware circuitry and software, nor to any particular source for the instructions executed by the processing unit.

Memory 34 contains instructions for directing processing unit 32 to carry out the example method 100 outlined in FIG. 2. As indicated by block 102 and FIG. 2, instructions stored in memory 34 direct processing unit 32 to detect and identify acoustical contact (AC) between transducer sensing area 26 and the anatomy or body being examined. In one implementation, processing unit 32 determines acoustical contact between transducer sensing area 26 and the anatomy or body being examined based upon signals received from transducer sensing area 26 of probe 22.

In one implementation, instructions in memory 34 direct processing unit 32 to calculate or determine a frequency spectrum which is used to determine acoustic contact of transducer sensing area 26 of probe 22 with the object or anatomy being examined. As described in more detail in U.S. Pat. No. 8,002,704 which issued on Aug. 23, 2011 to Torp et al., the full disclosure of which is hereby incorporated by reference, processor 32 receives RF scanline data or complex demodulated RF scanline data from an image sector generated by ultrasound system 20. In operation, and for example, for a probe having a 1-D array transducer, the lateral frequency spectrum is equal to the two-way aperture function in the focal plane. This results from the Fraunhofer approximation and the assumption of linear propagation of pressure waves. Further, the two-way aperture function is given by the convolution of the transmit and receive aperture functions. A typical example of equal size transmit and receive apertures with rectangular apodization results in a triangular shaped amplitude-spectrum with a bandwidth proportional to the sum of the transmit and receive aperture as described herein. In general, the spectral shape and size is given by the aperture functions.

There is ideally a one-to-one mapping between the frequency spectrum and the autoconvolution of the probe aperture function. Comparing the Fourier transformed data to the two-way probe aperture function is performed and identifies regions with reduced spectral amplitude that correspond to regions on the aperture with improper contact. It should be noted that the use of frequency spectrum to detect acoustical contact may be implemented in different types of phased array probes, including, for example, probes having 2-D arrays, wherein the frequency spectrum in both the azimuth and elevation direction will provide an image of the two-dimensional aperture function. Additionally, the amplitude spectrum in the radial direction may be calculated and visualized. This 2-D (for 1-D arrays) or 3-D (for 2-D arrays) spectrum also has information relating to image resolution in the radial direction, showing for example, the amount of frequency dependent attenuation present at the current probe position.

The received data is processed to provide a lateral map. The lateral map is a collection of a single Fourier coefficient from a radial Fourier transform of each beam produced by the probe. More particularly, band pass filtering of the data is performed around the pulse demodulation frequency. For IQ demodulated data, this filtering simplifies to averaging (or summing) radial samples along each beam that corresponds to the low pass filtering. In operation, the more radial samples included in the summation, the narrower the filter frequency response. Specifically, a spatial frequency response 200 of the ultrasound or other imaging system is determined and which may be used to indicate image quality or acoustic contact. As shown in FIG. 3, the spatial frequency response is calculated in all three dimension in the k-space, namely kₓ, k_{y} and k_{z}. The spatial frequency response (4π/Δ) of a slice 202 defining the center of central frequency is calculated as is known wherein B_{w} defines the radial bandwidth and [symbol] defines the wavelength of an emitted pulse. Thus, the spectral band/plane is calculated in the k-space.

The lateral frequency spectrum is then calculated by Fourier-transforming the averaged IQ-signal. The absolute value of the spectrum is then shifted to center the zero-frequency component. Thus, the left portion of the spectrum corresponds to the left side of the probe and the right side of spectrum corresponds to the right side of the probe. It should be noted that a Fast Fourier Transform algorithm may be implemented to reduce the processing time. Further, it should be noted that various embodiments are not limited to Fourier transforming, but different processing may be performed, for example, parametric frequency spectrum analysis.

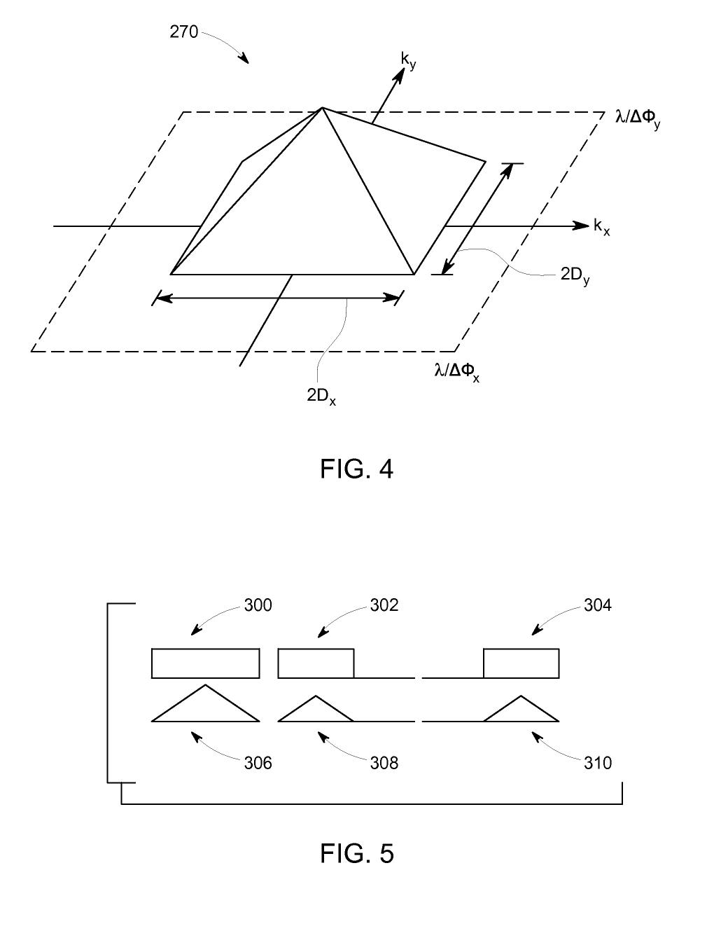

Referring now to FIG. 4 illustrating a lateral frequency spectrum 270 showing a triangular two-way aperture function in linear scale, assuming the probe aperture is centered around zero with a width D, then the corresponding Nyquist range is expressed as follows: [symbol] /Δγ, where Δγ is the beam sampling density in radians and [symbol] is the wavelength of the transmitted pulse.

For second harmonic (octave) imaging, [symbol] is the wavelength corresponding to twice the transmitted frequency, which is shown in FIG. 5 wherein the x-axis is scaled in meters. Further, values outside D do not correspond to a part of the aperture of the probe, but are indicative of side lobes in the spectrum. If a smooth window function is used prior to the Fourier transform, the side lobes are low (e.g., −40 dB for a Hamming window). In operation, increased side lobe levels indicate the presence of unwanted signal components (e.g., reverberation noise).

Temporal and spatial averaging then may be applied to reduce the variance in the spectrum estimates. For example, successive frequency spectrum images are averaged temporally from frame to frame. Spatially, each frequency spectrum is smoothed by low pass filtering. Alternatively, the available radial samples are divided into separate segments, with each producing a spectral estimate, and which are then averaged to produce one final spectrum estimate. Various known methods of frequency spectrum estimation may be used, for example, the Welch method of power spectrum estimation. Dynamic compression is then performed. Specifically, in one embodiment, dynamic compression in the form of a logarithmic transform provides visualization of a range of intensities without clipping weak signals. Signal strength in ultrasound imaging may vary, for example, due to different types of tissue having varying ability to reflect ultrasound.

Gain control is then performed. In operation using the ultrasound system 100 or 150, different settings and examination of different types of tissue result in different signal intensities. Gain control is used to normalize spectrum amplitude. In one embodiment, manual gain control is provided via a user input device. Specifically, a user may set the gain and dynamic range of the displayed spectrum. In other embodiments, automatic gain control may be provided using a gain control algorithm as is known.

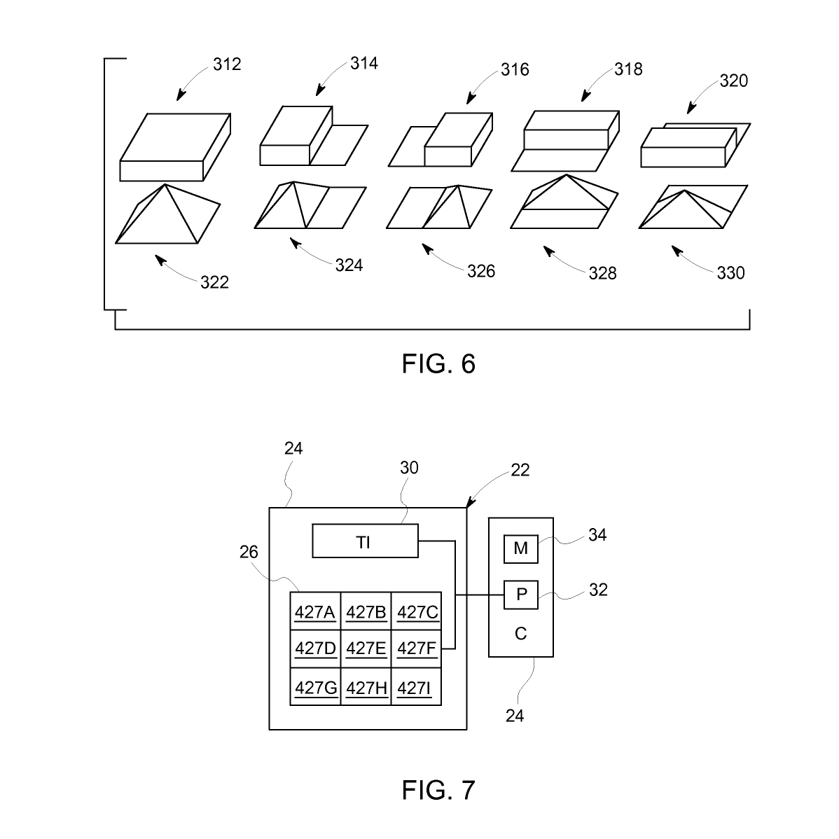

The lateral frequency spectrum is then visualized based on the type of probe. For example, as shown in FIGS. 5 and 6, probes having a one-dimensional aperture generate a one-dimensional lateral spectrum that may be visualized. Probes having a two-dimensional aperture generate a two-dimensional lateral spectrum that may be visualized in the form of, for example, a color-coded two-dimensional contact map. In particular, as shown in FIG. 5, for linear phased array probes, the aperture contacts 300, 302 and 304 result in lateral spectrums 306, 308 and 310, respectively. Further, as shown in FIG. 6, for a two dimensional matrix probe, the aperture contacts 312, 314, 316, 318 and 320 result in lateral spectrums 322, 324, 326, 328 and 330, respectively. This “visualization” constitutes data regarding acoustic contact for different portions of transducer sensing area 26.

In other implementations, instructions in memory 34 direct processing unit 32 to determine or detect acoustical contact using signals from transducer sensing area 26 in other manners. As indicated by broken lines in FIG. 1, in still other implementations, probe 22 comprises one or more sensors 38, in addition to transducer sensing area 26, which sense parameters and output signals that indicate acoustical contact of transducer sensing area 26 with the anatomy or object being examined. For example, sensor 38 may comprise one or more pressure sensing elements or electrical capacitive contact sensors located along, around or interspersed amongst the individual ultrasound sensing elements or aperture contacts of transducer sensing area 26. In one implementation, sensor 38 detects an overall degree are extended acoustical contact between the entire transducer sensing area 26 and the anatomy or object being examined. In yet another implementation, sensor 38 has greater resolution, outputting signals that indicate acoustical contact between individual portions of transducer sensing area 26 and the object or anatomy being examined. For example, sensor 38 may comprise a row or a two-dimensional array of multiple individual sensing elements that indicate different extensive acoustical contact for different regions or portions of transducer sensing area 26.

As indicated by block 104 of method 100 shown in FIG. 2, instructions contained in memory 34 direct processor 32 to output control signals to actuate tactile indicator 30 based upon the detected acoustical contact. In one implementation, instructions in memory 34 direct processor 32 to output control signals to actuate tactile indicator 32 one of a plurality of available or selectable states based upon an overall acoustical contact valuation for the entire transducer sensing area 26. For example, in one implementation, controller 24 determines a general level of acoustical contact for the entire transducer sensing area 26 and actuates tactile indicator 30 based upon the general level of acoustical contact for the entire transducer sensing area 26. In such an implementation, controller 24 may not identify acoustical contact differences between different portions of transducer sensing area 26.

In yet another implementation, controller 24 identifies acoustical contact differences between different portions of transducer sensing area 26 and utilizes the different acoustical contact values for the different portions to output a single homogenous tactile feedback. As illustrated by FIG. 7, controller 24 determines an extent or degree of acoustical contact for each of a plurality of distinct portions 427 of transducer sensing area 26. An individual sensing portion 427 comprises a subset (less than all) of the total number of sensing elements, contact apertures, of transducer sensing area 26. In one implementation, individual sensing portion 427 comprises a predefined cluster or group of adjacent sensing elements or contact apertures. In another implementation, an individual sensing portion 427 may consist of an individual sensing element or contact aperture. As noted above with respect to FIGS. 3-6, spectrum frequency may be utilized to identify acoustical contact for each of the plurality of distinct portions of transducer sensing area 26.

In the example illustrated in FIG. 7, controller 24 provides the caretaker with a single homogenous feedback which is based upon an overall acoustical contact value derived from an aggregate of individual acoustical contact values for the different portions 427 of transducer sensing area 26. For example, in one implementation, controller 24 calculates a statistical value across an entire area of transducer sensing area 26 using each of the individual acoustical contact values for each of the individual portions 427. In one implementation, controller 24 determines or calculates an average quantified degree of acoustical contact of all of the aperture contacts of transducer sensing array 26. In such an implementation, processor 32 outputs control signals actuating tactile indicator 30 to one of a plurality of selected states based upon the aggregate of individual acoustical contact values determined for the individual portions 427.

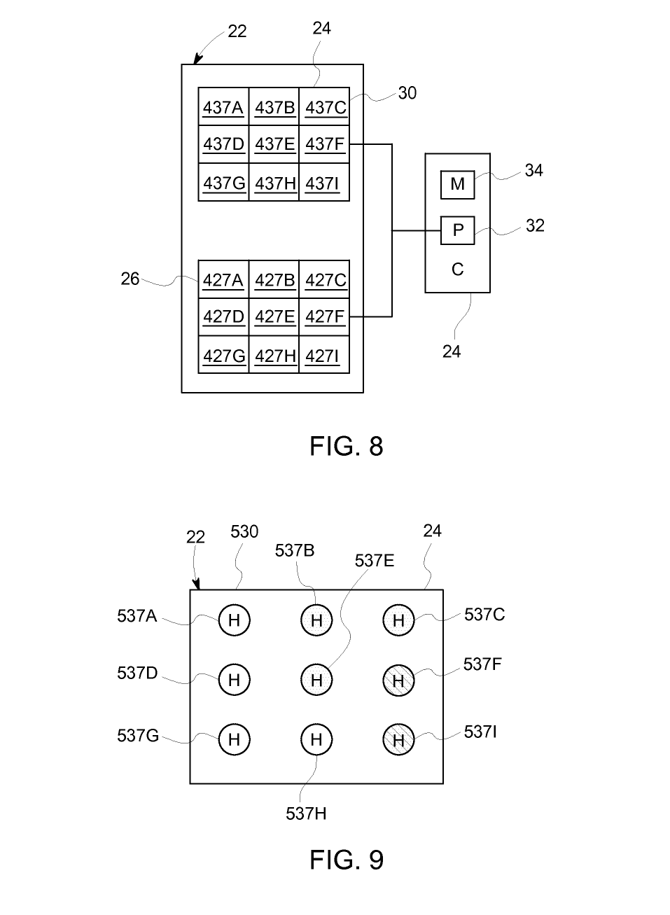

In yet another implementation, controller 24 determines acoustical contact values for each of the plurality of different sensing portions 427 and outputs control signals to provide tactile feedback indicating the different individual levels of acoustical contact for each of the portions 427 of transducer sensing area 26. In the example illustrated in FIG. 8, tactile indicator 30 comprises a plurality of distinct tactile elements or tactile portions 437A-437I (collectively referred to as tactile portions 437) along surface 24 of probe 22. In one implementation, each of such tactile portions 437 are physically located on outer surface 24 at a location relative to other tactile portions 437 based upon the location of the associated sensing portion 427 relative to other sensing portions 427 of transducer sensing area 26. For example, tactile sensing portion 427A is in the upper left corner of transducer sensing area 26. Accordingly, tactile portion 437A, which is to indicate acoustical contact for sensing portion 427A, is also in the upper left hand corner of tactile indicator 30.

In one implementation, each individual tactile portion 437 is additionally sized and/or shaped proportional to the size and/or shape of the individual sensing portion 427 being represented by the tactile portion 437. For example, transducer sensing area 26 may comprise two sensing portions 427 which have different shapes and/or have different sizes. In such an implementation, the tactile portions 437 assigned to the two sensing portions 427 would have similar differences in shape and similar proportional differences in size.

In the implementation illustrated in FIG. 8, controller 24 directs processor 32 to differently actuate each of the individual tactile portions 437 based upon the acoustic contact properties are acoustical contact values of their respective assigned sensing portions 427. For example, in one implementation, controller 24 compares the acoustical contact value for each individual sensing portion 427 with one or more predefined thresholds, wherein controller 24 actuates each individual corresponding tactile portion 437 to one of a plurality of different tactile states based upon the comparison. In yet another implementation, controller 24 actuates each individual tactile portion 437 to one of a plurality of states in direct proportion to the individual acoustical contact value identified for the corresponding sensing portion 427.

Although FIG. 7 illustrates nine sensing portions 427 and although FIG. 8 illustrates nine sensing portions 427 and a corresponding nine tactile portions 437, in other implementations, probe 22 may comprise a greater or fewer of such sensing portions 427 and a greater or fewer of such tactile portions 437. Although FIGS. 7 and 8 schematically illustrate portions 427 and 437 arranged a two-dimensional grid or box, in other implementations, portions 427 and 437 may be arranged in other shapes and configurations. For example, in another implementation, portions 427 and 437 may be arranged in other oval, circular, irregular or other polygonal shapes. In one implementation, transducer sensing area 26 is partitioned into a plurality of concentric rings, wherein tactile indicator 30 comprises tactile portions 437 also arranged in a plurality of concentric rings.

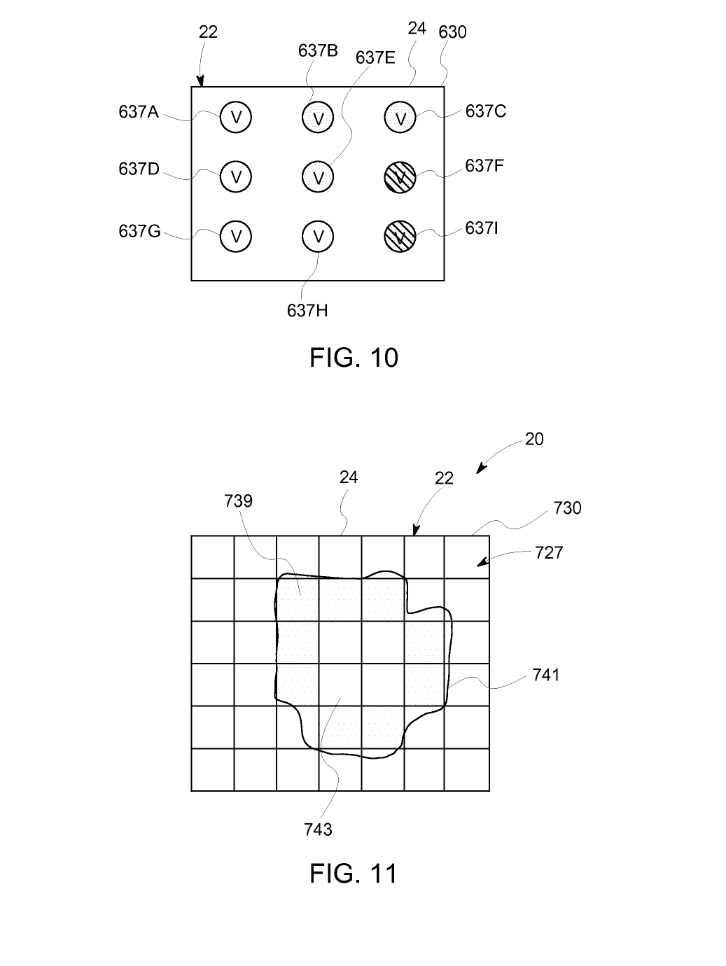

FIGS. 9 and 10 illustrate two example tactile indicators extending along surface 24 of probe 22. FIG. 9 illustrates tactile indicator 530, an example implementation of tactile indicator 30. Tactile indicator 530 comprises a two-dimensional array of tactile portions 537A-537I (collectively referred to as tactile portions 537). Tactile portions 537 correspond to sensing portions 427 shown in FIGS. 7 and 8. In the example illustrated, each of tactile portions 537 comprises a heating element, such as a resistor, which is actuatable to different heat emitting states.

FIG. 9 illustrates one example of tactile feedback provided by tactile indicator 530. As indicated by no stippling or crosshatching, controller 24 has actuated tactile portions 537A, 537D, 537G and 537H to a first heat emitting state or temperature based upon the acoustical contact values for the corresponding sensing portions 427A, 427D, 427G and 427H. As indicated by stippling, controller 24 has actuated tactile portions 537B, 537C, 537E to a second heat emitting state or temperature, different than the first heat emitting state or temperature, based upon different determined acoustical contact values for sensing portions 427B, 427C, 427E, respectively. As indicated by stippling and crosshatching, controller 24 has actuated tactile portions 537F and 537I to a third heat emitting state or temperature, different than both the first heat emitting state and the second heat emitting state, based upon the different determined acoustical contact values for sensing portions 427F and 427I, respectively. The different temperature states provide tactile feedback to the person gripping or holding probe 22; the temperature states indicating different degrees acoustical contact for the different individual sensing portions 427 of transducer sensing area 26. For example, such different temperatures may indicate that sensing portions 427A, 427D, 427G and 427H have a poor level of acoustical contact, while sensing portions 427B, 437C and 437E have an average or acceptable level of acoustical contact and that sensing portions 427F and 427G have a superior or excellent level or degree of acoustical contact. As a result, the person gripping 24 may utilize such feedback to appropriately reposition probe 22 to acquire enhanced imaging results, such as increasing the level of acoustical contact for all the different sensing portions or achieving a greater number of or percentage of sensing portions having level of acoustical contact.

FIG. 10 illustrates tactile indicator 630, another example implementation of tactile indicator 30. Tactile indicator 630 comprises a two-dimensional array of tactile portions 637A-637I (collectively referred to as tactile portions 637). Tactile portions 637 correspond to sensing portions 427 shown in FIGS. 7 and 8. In the example illustrated, each of tactile portions 537 comprises a vibrating element, such as a vibration motor, which is actuatable to different vibrating states.

FIG. 10 illustrates one example of tactile feedback provided by tactile indicator 630. As indicated by no stippling or crosshatching, controller 24 has actuated tactile portions 637A, 637D, 637G and 637H to a first vibrating state based upon the acoustical contact values for the corresponding sensing portions 427A, 427D, 427G and 427H. As indicated by stippling, controller 24 has actuated tactile portions 637B, 637C, 637E to a second vibrating state, different than the first vibrating state, based upon different determined acoustical contact values for sensing portions 427B, 427C, 427E, respectively. As indicated by stippling and crosshatching, controller 24 has actuated tactile portions 637F and 637I to a third vibrating state, different than both the first vibrating state and the second vibrating state, based upon the different determined acoustical contact values for sensing portions 427F and 427I, respectively. In one implementation, one of the “vibrating states” is a level of zero or no vibration. The different vibrating states provide tactile feedback to the person gripping or holding probe 22; the vibrating states indicating different degrees of acoustical contact for the different portions of transducer sensing area 26. As a result, the person gripping 24 may utilize such feedback to a properly reposition probe 22 to acquire enhanced imaging results.

In the example illustrated in FIGS. 9 and 10, each of the sensing portions 427 having a similar determined extent of acoustic contact with the anatomy or object being examined is represented by a corresponding tactile portion 437 actuated to a state based upon the determined extent of acoustic contact. FIG. 11 illustrates an alternative selectable mode for the operation of system 20. FIG. 11 illustrates tactile indicator 730, another implementation of tactile indicator 30. Tactile indicator 730 comprises tactile portions 737 which correspond to individual sensing portions of transducer sensing area 26. In the mode of operation illustrated in FIG. 11, controller 24 actuates selected tactile portions 737 to haptically indicate boundaries or a perimeter of a cluster or group of sensing portions having the same or similar (within a predefined range) acoustic contact properties. In the example illustrated, controller 24 actuates perimeter tactile portions 739 (indicated by stippling) to a tactile state different than adjacent tactile portions 737. Tactile portions 739 define the boundary of a larger region 741 having the same or similar acoustic contact properties. As shown by FIG. 11, perimeter tactile portions 739 surround or extend about central or intermediate tactile portions 743 which have different tactile properties as compared to tactile portion 739. In the mode illustrated in FIG. 11, system 20 haptically indicates the boundary of a cluster or group of sensing portions having the same or similar acoustical contact characteristics.

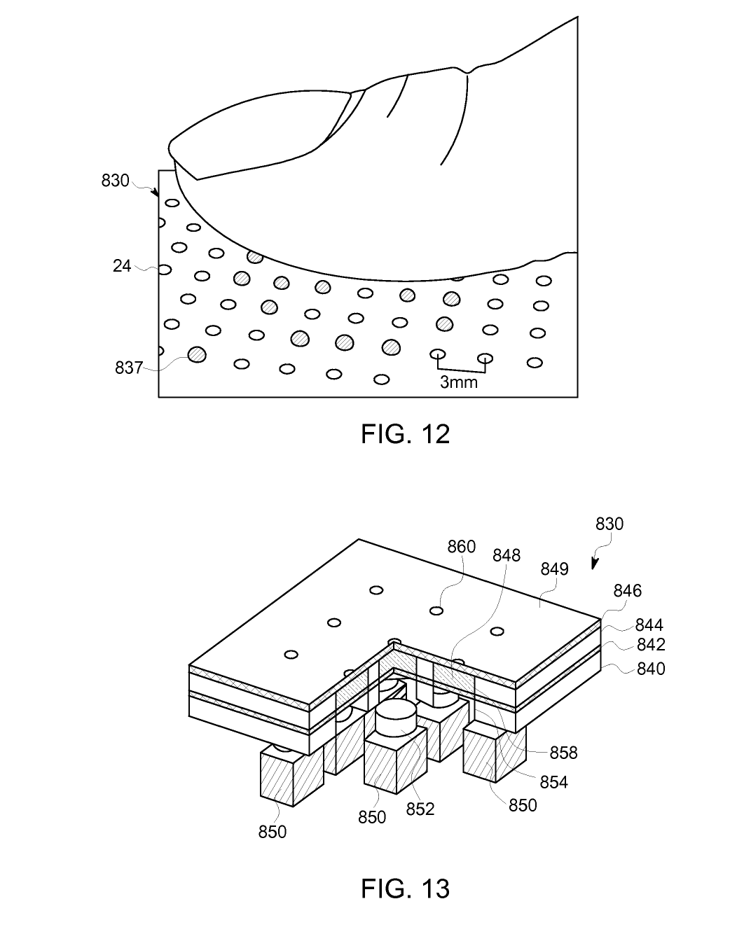

FIGS. 12 and 13 illustrate tactile indicator 830, another implementation of tactile indicator 30. As shown by 12, tactile indicator 830 comprises surface 24 having a two-dimensional array of individually actuatable tactile portions 837. Tactile portions 837 are selectively raised and lowered to provide tactile or haptic feedback regarding acoustic contact properties of the corresponding are associated sensing portions of transducer sensing area 26 (shown in FIG. 8).

FIG. 13 is a sectional view of one example implementation of tactile indicator 830. In the example illustrated, tactile indicator 830 comprises a substrate 840, diaphragm 842, spacer layer 844, tactile layer 846, fluid 848, cover layer 849 and actuators 850. Substrate 840 comprise a base layer underlying supporting the remaining layers. Substrate 840 comprises openings 854 through which actuators 850 influence or move portions of diaphragm 842. In one implementation, substrate 40 comprise a rigid polymer such as poly methyl methacrylate (PMMA). In other implementations, substrate 40 may comprise other materials.

Diaphragm 842 comprises a layer of resiliently flexible material extending across openings 854 in substrate 40. Diaphragm 842 is configured to be pushed upwardly by actuators 850 displace fluid 854. In one implementation, diaphragm 842 comprises a deformable polymer such as poly dimethyl siloxane (PDMS). In other implementations, diaphragm 842 may comprise other deformable polymers or other rubber-like films or membranes.

Spacer layer 844 extends above diaphragm 842 and cooperates with tactile layer 846 to form chambers 858. Spacer layer 844 is formed from a material and/or has a thickness so as to not vendor flex as actuators 850 the form layers 842 and 846. In one implementation, spacer layer 844 comprises a somewhat rigid polymer such as poly methyl methacrylate (PMMA). In other implementations, spacer layer 844 may comprise other materials.

Tactile layer 846 comprises a layer, film or membrane of material configured to resiliently deform and bulge through and above openings 860 in cover layer 849 to form and provide tactile portions 837. In one implementation, tactile layer 846 comprises a highly deformable polymer such as poly dimethyl siloxane (PDMS). In other implementations, diaphragm 842 may comprise other deformable polymers or other rubber-like films or membranes.

Fluid 848 comprises a liquid or gas captured within each of chambers 858 which are defined by diaphragm 842, spacer layer 844 and tactile layer 846. Fluid 848 transmits motion of diaphragm 842 to tactile layer 846 to move tactile layer 846 through opening 860 the last to extend above or below cover layer 849. In one implementation, fluid 848 comprises glycerin. In other implementations, fluid 848 may comprise other liquids or gases. In some implementations, rigid mechanical structures, such as pins, are used in place of fluid 848 to transmit force from actuators 850 to tactile layer 846 so as to displace portions of tactile layer 846 through openings 8602 form tactile portions 837 (shown in FIG. 12).

Cover layer 849 comprise a layer of material configured so as to at a lower level of flexibility as compared to tactile layer 846. Cover layer 849 maintained its shape at tactile layer 846 is deformed and pushed through opening 860 in cover layer 849. In one implementation, cover layer 849 forms the outer surface 24 of portions of probe 22. In one implementation, cover layer 849 is formed from a rigid polymer. In yet other implementations, cover layer 849 supports additional other overlying layers of material which may be soft, compressible or flexible.

Actuators 850 comprise individually actuatable devices located and configured to interact with diaphragm 842 through openings 854 so as to raise and lower portions of diaphragm 842 so as to raise and lower portions of tactile layer 846 through openings 860 to selectively form tactile portions 837 shown in FIG. 12. In one implementation, controller 24 (shown in FIG. 8) generates control signals causing actuators 850 to actuate tactile portions 837 of tactile indicator 830 to different heights based upon acoustic contact properties of associated or corresponding sensing portions. In one implementation, each of actuators 850 comprises a piezo electric actuator having a piston 852 which is selectively raised and lowered against diaphragm 842. In yet other implementations, each of actuators 850 comprises other types of mechanisms for selectively raising and lowering distinct portions of diaphragm 842 to individually and selectively raise and lower portions of tactile layer 846 through opening 860 to selectively adjust the state of each of tactile portions 837 shown FIG. 12.

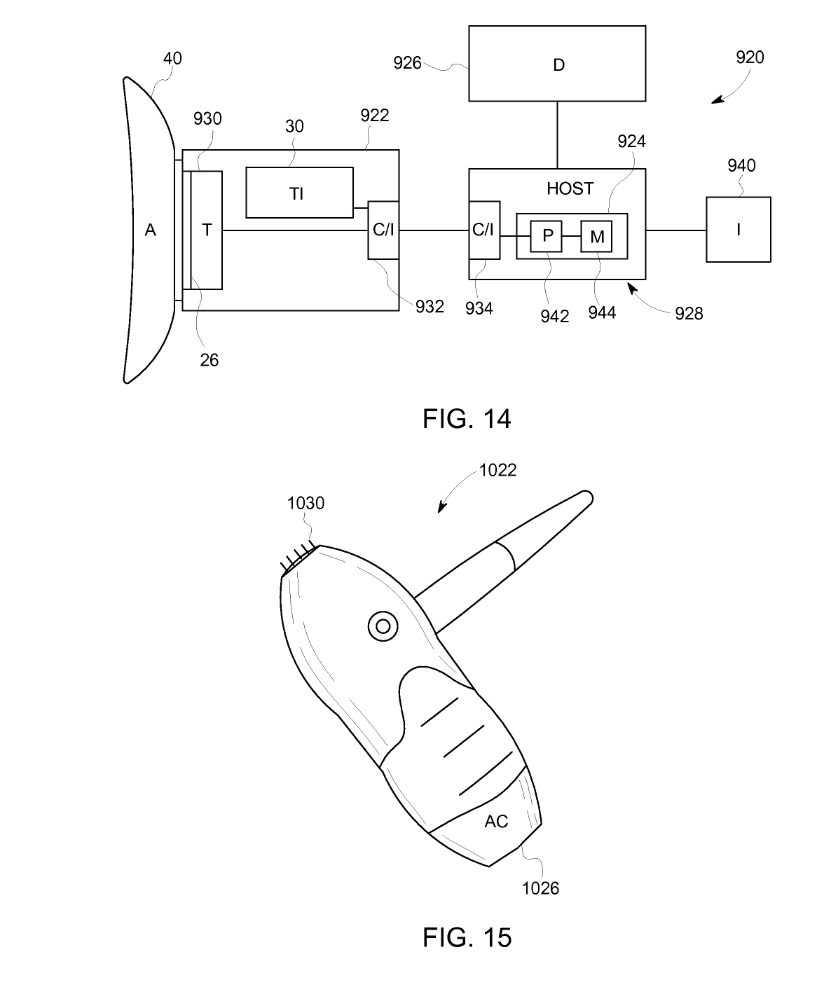

FIG. 14 schematically illustrates an example ultrasound system 920, an example implementation of ultrasound system 20. Ultrasound system 920 comprises probe 922, input 924, display 926 and host 928. Probe 922 comprises a handheld instrument by which ultrasound waves or pulses are directed into anatomy 40 and by which reflections of such waves are sensed to produce signals which are transmitted to host 28. Probe 922 provides tactile feedback regarding acoustic contact, permitting a physician or caretaker to focus his or her attention on the patient. Probe 922 comprises a transducer 930 having transducer sensing area 26; tactile indicator 30; and communication interface 932. Transducer sensing area 26 and tactile indicator 30 are described above with respect to FIGS. 1-13.

Communication interface 932 comprises an interface by which probe 922 communicates with host 928. In one implementation, communication interface 932 facilitates wireless communication. For example, in one implementation, communication interface 932 comprises a wireless antenna. In another implementation, communication interface may comprise optical communication technology, such as an infrared transmitter. In another implementation, communication interface 932 facilitates a wired communication such as through a cable. For example, communication interface 932 may comprise a USB port or other communication port.

Input 924 comprises a device by which a person may provide selections, commands or instructions to host 928. Input 924 may comprise a keyboard, a mouse, a microphone with speech recognition software, a keypad and the like. Input 924 may be incorporated as part of a monitor which provides host 928. Input 924 may also be incorporated as part of display 926, wherein display 926 comprises a touch screen. Alternatively, input 924 may comprise one or more separate input structures in communication with host 928 in a wired or wireless fashion. In some implementations, input 924 may be omitted.

Display 926 comprises a screen or other display by which the results from probe 922 are visibly presented to a caretaker, such as a doctor or nurse. In one implementation, display 926 may comprise a separate screen distinct from host 28 and in communication with host 928 in a wired or wireless fashion. In another implementation, display 926 may be incorporated as part of host 928 as part of a single self-contained unit.

Host 928 comprises a monitor or other unit which analyzes signals from probe 922 and presents the results of the analysis as well as the signals themselves on display 926. In the example illustrated, host 928 additionally controls tactile indicator 30 of probe 922. Host 928 comprises communication interface 934 and controller 940. Communication interface 934 comprises an interface by which host 928 communicates with probe 922. In one implementation, communication interface 934 facilitates wireless communication. For example, in one implementation, communication interface 934 comprises a wireless antenna. In another implementation, communication interface may comprise optical communication technology, such as an infrared transmitter. In another implementation, to communication interface 934 facilitates a wired communication such as through a cable. For example, communication interface may comprise a USB port or other communication port.

Controller 940 comprises processor 942 and memory 944. According to one implementation, Prosser 942, following instructions contained in memory 944, receives ultrasound echo signals from probe 922 and analyzes such signals, wherein the results of such analysis are presented on display 926. In one implementation, controller 940 comprises circuitry providing beam former, radiofrequency (RF) processor and signal processor. Such circuitry causes probe 922 to emit ultrasound signals, receives ultrasound signals or echoes and generates ultrasound images based upon such ultrasound echoes.

In the example illustrated, controller 940 further functions similar to controller 24 described above. In particular, controller 940 carries out method 100 shown in FIG. 2. Controller 940 detects and identifies acoustical contact (AC) between transducer sensing area 26 and the anatomy or body being examined. In one implementation, controller 940 determines acoustical contact between transducer sensing area 26 and the anatomy or body 40 being examined based upon signals received from transducer sensing area 26 of probe 22. Controller 940 further outputs control signals to actuate tactile indicator 30 based upon the detected acoustical contact. In a first selected mode of operation, controller 940 outputs control signals to actuate tactile indicator 32 one of a plurality of available or selectable states based upon an overall acoustical contact valuation for the entire transducer sensing area 26. In a second selected mode of operation, controller 940 provides the caretaker with a single homogenous feedback which is based upon an overall acoustical contact value derived from an aggregate of individual acoustical contact values for the different portions of transducer sensing area 26. In a third selected mode of operation, controller 24 determines acoustical contact values for each of the plurality of different sensing portions and outputs control signals to provide tactile feedback indicating the different individual levels of acoustical contact for each of the portions of transducer sensing area 26. In one implementation, each of the above-described feedback modes are selectable by the caretaker user through input 940.

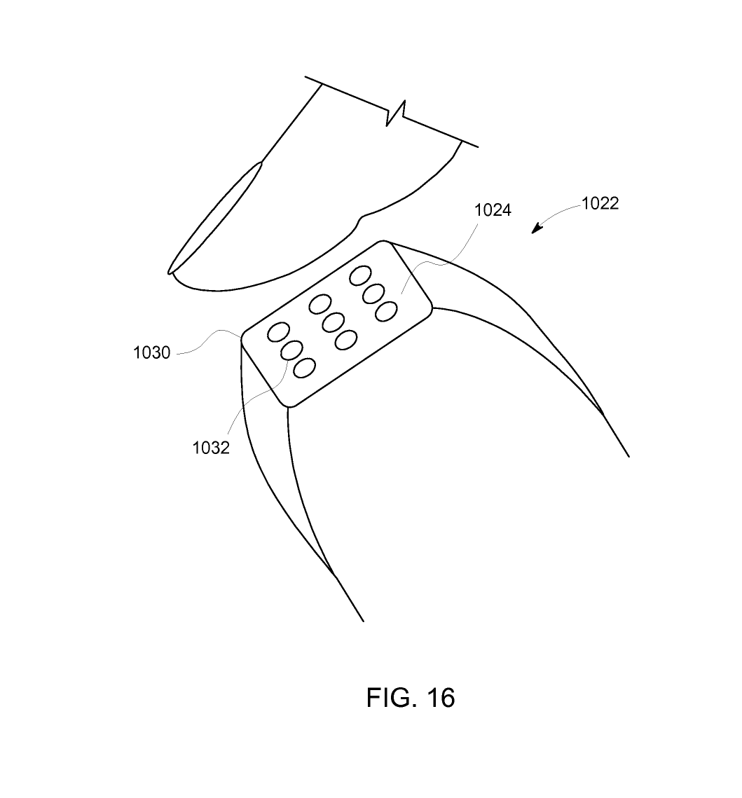

FIGS. 15 and 16 illustrate probe 1022, an example implementation of probe 22, 922. As shown by FIG. 15, probe 1022 comprises transducer sensing area 1026 and tactile indicator 1030, example implementations of transducer sensing area 26 and tactile indicator 30 described above. FIG. 16 is a perspective view illustrating tactile indicator 1030. In the example illustrated, tactile indicator 1030 comprise a two-dimensional grid or array of openings 1032 through which projections, pins or structures may be actuated to different heights to indicate acoustic contact characteristics or properties of corresponding sensing portions of transducer sensing area 1026. In one implementation, tactile indicator 1030 is similar to tactile indicator 830 described above in FIGS. 12 and 13. In yet other implementations, tactile indicator 1030 may have other configurations.

In operation, a person, user or caretaker manually contacts surface 1024, including tactile indicator 1030, while manipulating probe 1022 and positioning probe 1022 against the anatomy 40 being examined. As the person manipulates probe 1022 and repositions probe 1022, he or she receives different tactile or haptic sensations along surface 1024. Such haptic sensations correspond to the degree to which different sensing portions of transducer sensing area 1026 are in acoustic contact with the anatomy 40. Using such feedback, the person may manually manipulate probe 1022 to an appropriate orientation and position at which acoustic contact is enhanced for enhanced ultrasound image quality. Because such feedback regarding acoustic contact is communicated through touch, the caretaker person may maintain his or her focus on the patient during the examination.

Although systems 20 and 920 are described above that providing tactile feedback regarding acoustic contact between a transducer sensing area in the anatomy or object being examined, in other implementations, systems 20 and 920 provide tactile feedback regarding other parameters associated with the use of ultrasound probe 22, 922. In one implementation, systems 20 and 920 provide tactile feedback indicating current operational parameters or settings under which systems 20, 920 are operating. In another implementation, systems 20, 920 provide tactile feedback indicating performance levels or performance parameters (such a signal to noise ratio) currently being attained by the ultrasound system. In another implementation, systems 20 and 920 provide tactile feedback regarding a sensed, detected or determined relationship between systems 20, 920 and the anatomy and/or object being examined. Providing tactile feedback regarding acoustic contact between a transducer sensing area of probe 22, 922 and the anatomy or object being examined is just one example of providing tactile feedback regarding the relationship between the ultrasound system 20, 920 and the anatomy or object being examined. In other implementations, systems 20, 920 provide tactile feedback regarding the relationship between ultrasound system 20, 920 and a target portion of the anatomy or object being examined. For purposes of this disclosure, a “parameter” of the ultrasound probe comprises of the current operational parameter setting under which an ultrasound system is operating, a performance level or levels currently being attained by the ultrasound system and/or a relationship of the ultrasound system and an anatomy/object being examined. A “parameter(s) may comprise (1) a static parameter describing the (physical) probe characteristics, such as the frequency range or (2) a parameter deduced/generated/estimated by processing the ultrasound signals received by the ultrasound probe.

FIG. 17 schematically illustrates ultrasound system 1120, another example of ultrasound system 20. Ultrasound system 1120 is similar to systems 20 and 920 described above except that system 1120 is configured to operate in an additional mode in which system 1120 provides tactile feedback regarding the relationship between system 1120 and a target portion of an anatomy or object being examined. As with system 20, system 1120 comprises probe 22 comprising transducer sensing area 26 and tactile indicator 30, each of which is described above.

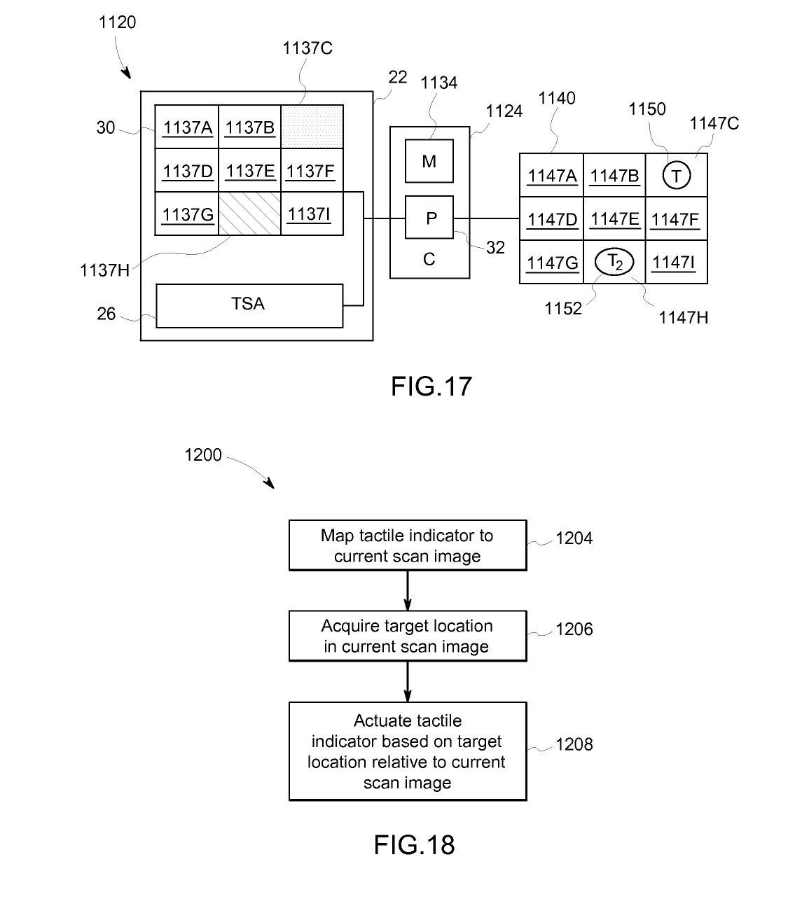

System 1120 further comprises controller 1124. Controller 1124 is similar to controller 24 except that controller 1124 comprises memory 1134 which includes software, code, circuitry or other program logic to direct processor 32 to operate in an additional mode in which system 1120 provides tactile feedback regarding the relationship between system 1120 and a target portion of an anatomy or object being examined. In the example illustrated, memory 1134 comprises program logic to direct processor 32 to carry out method 1200 outlined in FIG. 18.

As indicated by block 1204 of method 1200 of FIG. 18, controller 1124 maps tactile indicator 30 to the current scan image 1140 being acquired. In other words, distinct portions of tactile indicator 30 are assigned to corresponding portions of the current scan image 1140. In one implementation, such mapping is performed by assigning distinct portion of tactile indicator 30 to corresponding individual or groups of contact apertures of transducer sensing area 26 and the portion of image 1140 produced by the associated contact apertures. In another implementation, such mapping is formed by controller 1124 digitally partitioning image 1140 and assigning the digitally partitioned portions of image 1140 to corresponding portions of tactile indicator 30.

Although tactile indicator 30 is schematically illustrated as comprising a two-dimensional array or grid of nine tactile indicator portions 1137A-1137I which are each individually mapped to corresponding portions 1147A-1147I, respectively, of image 1140, in other implementations, tactile indicator 30 is partitioned into other layouts having a greater or fewer number of such tactile indicator portions, wherein image 1140 is also partitioned into a corresponding number and arrangement of image portions. Although tactile indicator 30 and image 1140 are both illustrated as being partitioned into a two-dimensional rectangular grid having rows and columns, in other implementations, tactile indicator 30 and image 1140 are partitioned into corresponding other layouts, such as a center tactile indicator and image portion and a series of rings of indicator portions and image portions extending about the center region.

As indicated by block 1206 in FIG. 18, controller 1124 acquires a target location in the current scan image 1140. The target location is the location of a target, such as target 1150 (shown in FIG. 17), in the current scan image 1140. For example, in one implementation, the target 1150 comprises a needle which has been inserted into an anatomy being scanned. In one implementation, target 50 comprises an organic, biological structure, or an implant or other structure, inserted into an anatomy and/or moving within the anatomy. In another implementation, the target 1150 comprises a desired image plane or anatomy to be scanned.

In one implementation, the target and its location are input by the caretaker. In another implementation, the target and its location or determined or identified by controller 1124 based upon digital analysis of the current scan image 1140. In one implementation, the target and its location are stationary or static, such as when the target 1150 comprises a particular anatomy or image plane to be scanned. In another implementation, the target in its location may be moving or dynamic, such as when the target is a needle, catheter or other structure being tracked.

As further shown by FIG. 17, in one implementation, controller 1124 acquires locations of more than one target in the current scan image 1140. In the example shown FIG. 17, controller 1124 has acquired the location of a second target 1152. For example, in one implementation, target 1152 comprises a particular anatomy while target 1150 comprises a needle or other implant, wherein the controller 1144 acquires the relative positions and distances between the two targets 1150, 1152. In still other implementations, more than two targets are acquired

As indicated by block 1208 in FIG. 18, controller 1124 actuates tactile indicator 30 based upon the target location relative to the current scan image 1140. In one implementation, controller 1124 actuates tactile indicator 30 between different states based upon whether the target 1150 is centered within image 1140. In one implementation, controller 1124 actuates tactile indicator 30 between different states based upon a degree that transducer sensing area 26 centered over target 1150. For example, in one implementation, controller 1124 actuates tactile indicator 30 between different tactile states the degree at which transducer sensing area 26 is centered over the target 1150 increases.

In another implementation where controller 1124 has acquired location of a plurality of targets within the scan image, controller 1124 actuates tactile indicator 30 between different states based upon a distance or spacing between the plurality of targets, such as the spacing between targets, 1150, 1152, a relative positioning (above, below, to the right, to the left) of the two targets 1150, 1152 and/or the degree to which the two targets are centered opposite the transducer sensing area 26. For example, in one implementation, controller 1124 actuates one or more of portions 1137 of tactile indicator 30 between different tactile states (different vibration levels, different temperatures and/or different heights and the like) as the two targets 1150, 1152 become closer to one another, become farther apart from one another, become aligned, contact one another or are collectively centered opposite transducer sensor area 26.

In yet another implementation, controller 1124 differently actuates a selected one of portions or a selected set of portions 1137 of tactile indicator 30 based upon which portion 1147 of image 1140 contains the target, such as target 1150. In such an implementation, controller 1124 identifies which of portions 1147 target 1150 is located. Controller 1124 then outputs control signals actuating the corresponding portion of tactile indicator 32 a different tactile state as compared to surrounding portions of tactile indicator 30. In the example illustrated in FIG. 17, target 1150 is located within image portion 1147C. As a result, controller 1124 outputs control signals actuating the corresponding portion 1137C of tactile indicator 32 a different tactile state as indicated by stippling. As a result, the caretaker manipulating or handling probe 22 is provided with tactile feedback with regard to the relative positioning of probe 22 and transducer sensing area 26 with respect to target 1150.

In modes of operation where a plurality of target locations have been acquired, controller 1124 identifies or determines which of portions 1147 contain the plurality of targets and outputs control signals actuating the corresponding tactile indicator portions 1137 to different tactile states as compared to surrounding portions 1137 that are assigned to image portions 1147 that do not contain targets. In the example illustrated in FIG. 17, controller 1124 determines that the second target 1152 is within image portion 1147H. As a result, controller 1124 outputs control signals actuating the corresponding portion 1137C of tactile indicator 32 a different tactile state as indicated by crosshatching. As a result, a caretaker manipulating or handling probe 22 is provided with tactile feedback regarding the relative positioning of probe 22 as well as the relative positioning of both targets 1150 and 1152.

In one mode of operation, those portions 1137 of tactile indicator 30 corresponding to image portions 1147 containing targets are actuated to a same tactile state. In another mode of operation, different portions 1137 of tactile indicator 30 corresponding to different image portions 1147 containing different targets are actuated to different tactile states. For example, in one implementation, controller 1124 actuates tactile indicator portion 1137C to a first tactile state different than surrounding tactile states, as indicated by stippling, and actuates tactile indicator portion 1137H to a second state also different than surrounding tactile states, but also different than the tactile state of portion 1137C. As a result, in such a mode of operation, system 1120 provides tactile feedback to the person handling or manipulating probe 22 so as to identify and distinguish between each of the multiple targets within the current scan image 1140.

In one implementation, system 1120 provides tactile feedback regarding positioning of a first target, an inserted needle, with respect to a second target, the current image or scan plane. In another implementation, system 1120 provides tactile feedback regarding the positioning of the first target, an inserted needle, with respect to a second target, a desired ultrasound imaging or scan plane. For example, such tactile feedback may indicate the degree to which the needle is aligned with or in proximity to the desired ultrasound imaging plane. In yet another implementation, system 1120 provides tactile feedback regarding the positioning of a first target, the current scan plane, relative to the positioning of a second target, the desired scan plane. Such tactile feedback may indicate the degree to which the current scan plane is aligned with or corresponds with the desired scan plane. Such feedback may be beneficial in auto scan plane detection applications.

In some implementations, systems 20, 920 and 1120 operate in additional selectable modes, wherein tactile indicator 30 is actuated to one or more different tactile states so as to provide the person with tactile feedback regarding how he or she should adjust positioning of the probe. In other words, instead of the tactile feedback indicating the location of a target or the relationship of a target to the current scan plane, systems 20, 920 1120 provide tactile feedback which directly instructs or directs the caretaker to manipulate the probe in a certain fashion. For example, in one implementation, such tactile feedback may indicate a direction in which the user should rotate the probe 22, 922 in order to achieve a desired scan plane.

In yet other implementations, systems 20, 920 and 1120 operate in additional selectable modes, wherein tactile indicator 30 is actuated to one or more different tactile states so as to provide the person with tactile feedback regarding current performance parameters being achieved. For example, in one implementation, program logic in memory 1134 directs processor 32 to output control signals actuating one or more of portions 1137 of tactile indicator 30 to different tactile states based upon the current signal-to-noise ratio for an ultrasound scan. In one implementation, signal-to-noise ratio for Doppler may be indicated by the number of portions of tactile indicator 30 but have a particular tactile state, such as a number of tactile portions that are elevated, vibrating, heated or the like. In yet other implementations, tactile indicator 30 is actuated to different tactile states by the caretaker with feedback regarding other performance parameters.

While the preferred embodiments of the subject matter have been illustrated and described, it will be appreciated that various changes can be made therein without departing from the spirit and scope of the disclosure. For example, although different example embodiments may have been described as including one or more features providing one or more benefits, it is contemplated that the described features may be interchanged with one another or alternatively be combined with one another in the described example embodiments or in other alternative embodiments. One of skill in the art will understand that the subject matter of the present disclosure may also be practiced without many of the details described above. Accordingly, it will be intended to include all such alternatives, modifications and variations set forth within the spirit and scope of the appended claims. Further, some well-known structures or functions may not be shown or described in detail because such structures or functions would be known to one skilled in the art. Unless a term is specifically and overtly defined in this specification, the terminology used in the present specification is intended to be interpreted in its broadest reasonable manner, even though may be used conjunction with the description of certain specific embodiments of the present disclosure.

What is claimed is:

1. An apparatus comprising:
an ultrasound probe comprising:
a transducer sensing area;
a surface to be manually contacted by a hand while the hand is manipulating the probe; and
a tactile indicator along the surface, the tactile indicator actuatable to one of a plurality of tactile states; and
a controller to actuate the tactile indicator to a selected one of the plurality of tactile states based on a parameter of the ultrasound probe.

2. The apparatus of claim 1, wherein the plurality of tactile states are selected from a group of states consisting of: different heights of at least one protuberance; different shapes formed by at least one protuberance; different temperatures; different directional movements of at least one protuberance; and different vibrations.

3. The apparatus of claim 1, wherein the parameter of the ultrasound probe comprises acoustic contact of the transducer sensing area and an anatomy or object being examined.

4. The apparatus of claim 2, wherein the transducer sensing area comprises different sensing portions, wherein the sensor detects acoustic contact for each of the different sensing portions and wherein tactile indicator changes between the plurality of tactile states based upon acoustic contact for the different sensing portions.

5. The apparatus of claim 4, wherein the tactile indicator comprises a plurality of tactile portions, wherein the controller is to differently actuate different ones of the plurality of tactile portions between different tactile states based upon differences in acoustic contact of the different sensing portions.

6. The apparatus of claim 5, wherein each of the plurality of tactile portions is associated with one of the different sensing portions and wherein each of the plurality of tactile portions is physically located on the outer surface at a location relative to other tactile portions of the plurality of tactile portions based upon a location of the associated sensing portion relative to other sensing portions of the different sensing portions.

7. The apparatus of claim 1, wherein the tactile indicator comprises bumps and wherein the plurality of tactile states comprises a plurality of heights for the bumps.

8. The apparatus of claim 1, wherein the parameter comprises a relationship of a target to a current scan image.

9. The apparatus of claim 1, wherein the parameter comprises positioning of a needle with respect to one of a current scan plane and a desired scan plane.

10. The apparatus of claim 1, wherein the parameter comprises positioning of a current scan plane relative to a desired scan plane.

11. A method comprising:
identifying a parameter associated with a handheld ultrasound probe; and
actuating a tactile indicator on a hand-contacted surface of the handheld probe based upon the parameter.

12. The method of claim 11, wherein the actuation of the tactile indicator comprises actuating the tactile indicator between one of a plurality of tactile states selected from a group of states consisting of: different heights of at least one protuberance; different shapes formed by at least one protuberance; different temperatures; different directional movements of at least one protuberance; and different vibrations.

13. The method of claim 12, wherein the parameter comprises acoustic contact of a transducer sensing area of the handheld ultrasound probe with an anatomy or object being examined.

14. The method of claim 13, wherein the transducer sensing area comprises different sensing portions, wherein the detection of acoustic contact detects acoustic contact for each of the different sensing portions and wherein tactile indicator changes between the plurality of tactile states based upon acoustic contact for the different sensing portions.

15. The method of claim 13, wherein the tactile indicator comprises a plurality of tactile portions, wherein the actuation of the tactile indicator comprises differently actuating different ones of the plurality of tactile portions between different tactile states based upon differences in acoustic contact of the different sensing portions.

16. The method of claim 13, wherein each of the plurality of tactile portions is associated with one of the different sensing portions and wherein each of the plurality of tactile portions is physically located on the surface at a location relative to other tactile portions of the plurality of tactile portions based upon a location of the associated sensing portion relative to other sensing portions of the different sensing portions.

17. The method of claim 13, wherein the tactile indicator comprises bumps and wherein the actuation of the tactile indicator comprises actuating the bumps to different heights based upon the detected acoustic contact.

18. The method of claim 17, wherein bumps correspond to different sub apertures of the transducer sensing area detected as being in acoustic contact with skin and wherein actuation of the bumps to different heights is based upon which sub apertures of the transducer sensing area are detected as being in acoustic contact with the skin.

19. The method of claim 11, wherein the parameter comprises a relationship of a target to a current scan image.

20. An apparatus comprising:
a non-transitory computer-readable medium containing program logic to direct a processor to:
receive signals indicating a parameter of a handheld probe; and
output signals to actuate a tactile indicator on a hand-contacted surface of the handheld probe based upon the parameter.

21. The apparatus of claim 20, wherein the actuation of the tactile indicator comprises actuating the tactile indicator between one of a plurality of tactile states selected from a group of states consisting of: different heights of at least one protuberance; different shapes formed by at least one protuberance; different temperatures; different directional movements of at least one protuberance; and different vibrations.

22. The apparatus of claim 20, wherein the parameter comprises the contact of a transducer sensing area of the handheld probe with an anatomy or object being examined.

23. The apparatus of claim 20, wherein the transducer sensing area comprises different sensing portions, wherein the signals indicate acoustic contact for each of the different sensing portions and wherein tactile indicator changes between the plurality of tactile states based upon acoustic contact for the different sensing portions.

24. The apparatus of claim 23, wherein the tactile indicator comprises a plurality of tactile portions, wherein the actuation of the tactile indicator comprises differently actuating different ones of the plurality of tactile portions between different tactile states based upon differences in acoustic contact of the different sensing portions.

25. The apparatus of claim 23, wherein each of the plurality of tactile portions is associated with one of the different sensing portions and wherein each of the plurality of tactile portions is physically located on the surface at a location relative to other tactile portions of the plurality of tactile portions based upon a location of the associated sensing portion relative to other sensing portions of the different sensing portions.

26. The apparatus of claim 20, wherein the tactile indicator comprises bumps and wherein the actuation of the tactile indicator comprises actuating the bumps to different heights based upon the acoustic contact.

27. The apparatus of claim 20, wherein the parameter comprises a relationship of a target to a current scan image.
